(19) United States
(12) Patent Application Publication
(10) Pub. No.: US 2017/0333009 A1
(43) Pub. Date: Nov. 23, 2017

(54) INSTRUMENTS USED IN COLLECTING FLUID SAMPLES

(71) Applicant: Oregon Health & Science University, Portland, OR (US)

(72) Inventor: David L. Simons, Portland, OR (US)

(21) Appl. No.: 15/599,387

(22) Filed: May 18, 2017

Related U.S. Application Data
(60) Provisional application No. 62/338,959, filed on May 19, 2016.

(51) Int. Cl. A61B 10/00; F04B 17/03; F04B 19/04; F04B 19/22; F04B 9/02; F16H 31/00; F16H 19/04

(52) U.S. Cl. CPC A61B 10/0045; F04B 19/22; F04B 17/03; F04B 9/02; F04B 19/04; F16H 19/04; F16H 31/001

(57) ABSTRACT

Disclosed are instruments used in collecting fluid samples from a subject such as, for example, aqueous humor samples. The instrument involves a linear actuator that, when engaged by the user via a switch, causes a syringe held within the instrument to collect a defined amount of sample. Kits comprising the instrument as well as methods of using the instrument to collect fluid samples are also disclosed.

### CROSS-REFERENCES TO RELATED APPLICATIONS

This application claims the benefit of U.S. Provisional Patent Application No. 62/338,959, filed May 19, 2016, titled “INSTRUMENTS USED IN COLLECTING FLUID SAMPLES”, which is incorporated herein by reference in its entirety.

### FIELD OF THE INVENTION

Generally, the field involves instruments and devices used in the collection of samples. In particular, the field involves instruments and devices that incorporate a syringe in the collection of samples from small reservoirs in the body.

### BACKGROUND OF THE INVENTION

Diagnostic anterior chamber taps are typically done in the setting of intraocular inflammation to test for ocular infection. In such procedures a needle is inserted into the anterior chamber of the eye and about half of the 200-300 μl volume of the aqueous humor in the anterior chamber is removed. Removal of an excessive volume of aqueous humor may result in complications. Most commonly, the procedure is performed by a team of two operators—one operator to hold a needle and syringe in one hand where the needle is accessing the anterior chamber while focusing a slit lamp in the other hand and a second operator to pull back the plunger of the syringe. The second operator is unable to directly observe either the eye or the needle tip and relies on communication with the first operator to determine when to stop pulling the plunger.

Another method of performing the procedure involves the use of a pipette and bulb attached to the needle. The pipette bulb is compressed prior to the needle entering the eye and released after entry into the eye. This procedure allows for a single operator to perform the procedure, but is difficult to control and raises the risk of collapse of the anterior chamber. Clearly, improved methods of performing anterior chamber taps are necessary.

### BRIEF SUMMARY OF THE INVENTION

Disclosed herein are instruments used in collecting fluid samples from a subject. In embodiments, the instrument includes: a linear actuator; a switch configured to allow a user to engage the linear actuator. In such embodiments, one engagement of the switch by the user causes the linear actuator to move a defined distance. The instrument is further configured such that the user can hold all or a portion of a housing of the instrument in a first hand and operate the switch with a second hand. In some embodiments, the first hand is at the end of a substantially extended arm and the second hand is at the end of a substantially flexed arm. In embodiments, the instrument further includes a first holder coupled to the linear actuator. The first holder is configured to accept a portion of a syringe plunger such that the movement of the linear actuator by the defined distance moves the syringe plunger the defined distance. The instrument further includes a second holder coupled to the housing. The second holder is configured to accept a portion of a syringe barrel is held immobile relative to the plunger such that a defined amount of the fluid sample is collected in the syringe barrel when the syringe plunger moves the defined distance. In addition, the instrument is sufficiently light that it can be held by the user in the first hand for at least 1 minute.

In embodiments, the instrument can include a source of electrical power such as a battery or AC current. In such embodiments, the linear actuator can include a worm gear, lead screw, or ball screw and where the switch controls a motor such as a stepper motor, servo motor, or DC motor. In such embodiments, the switch can engage the linear actuator through a mechanical cable release, an electrical cable, or an infrared beam. In other embodiments, the instrument lacks an external power source. In such embodiments, the linear actuator can include a rack and pinion and the switch can mechanically engage a ratchet/pawl mechanism. In still other embodiments, the instrument weighs 50 g or less. In still other embodiments, the defined amount of fluid sample is 5-50 μl. In still other embodiments, the defined amount of fluid sample is 10-30 μl.

Disclosed herein are instruments used in collecting fluid samples from a subject. The instrument includes a linear actuator comprising a linear gear and a first circular gear, a ratchet/pawl mechanism engaged with the first circular gear, an access to the ratchet/pawl mechanism configured to accept a cable release such that the cable release can engage with the ratchet/pawl mechanism, a first holder coupled to the linear gear where the first holder is configured to accept a portion of a syringe plunger, and a second holder coupled to a housing, where the second holder is configured to accept a portion of a syringe barrel and where the second holder is held immobile relative to the first holder. In such an instrument, the linear actuator is configured to increase the distance between the first holder and the second holder upon engagement of the switch by the user. In embodiments, the cable release is coupled to the access. In such embodiments, the cable release is of sufficient length that a user can hold the instrument in a first hand and operate the switch with a second hand where the first hand is at the end of an extended arm and the second hand is at the end of a flexed arm. In such embodiments, the cable release is at least 10 cm in length. In embodiments, the instrument further comprises a second circular gear engaged with the ratchet/pawl mechanism and the first circular gear. In embodiments, the instrument comprises a reset mechanism configured to return the second holder to a starting position.

Disclosed herein are kits used in the collection of a fluid sample from a subject. The kits include the disclosed instruments, a syringe comprising a plunger configured to engage the first holder, a barrel configured to engage the second holder, and a needle. In embodiments, the kits further include a cable release, where the cable release is configured to engage the access of the disclosed instruments. In embodiments, the needle of the syringe is 25 gauge or smaller.

Disclosed herein are methods of collecting a sample comprising aqueous humor from an anterior chamber of an eye of a subject. Such methods involve inserting a needle into the anterior chamber, where the needle is attached to a syringe. The plunger of the syringe is held by the first holder of the disclosed instrument and where the barrel of the syringe is held by the second holder of the disclosed instrument. The methods further include engaging the switch of the disclosed instrument, thereby collecting the sample comprising aqueous humor. The method can further involve inserting the syringe into the disclosed instrument. In embodiments, the instrument is held by a first hand of a user and the switch is operated by a second hand of the user. In other embodiments, the second hand of the user is used to operate a slit lamp in addition to operating the switch. In other embodiments, the methods involve engaging the switch a sufficient number of times to collect at least 100 μl of the sample. In other embodiments, the methods involve removing the syringe from the instrument and resetting the first holder to a starting position.

It is an object of the invention to provide an instrument that allows more efficient collection of aqueous humor samples from the eye of a subject.

It is an object of the invention to allow a single user to collect aqueous humor samples from the eye of a subject while operating a slit lamp.

It is an object of the invention to provide a safer methodology for collecting aqueous humor samples from the eye of a subject that does not rely on communication between one medical professional looking into the eye using a slit lamp and holding a syringe and a second medical professional drawing fluid into the syringe.

It is an object of the invention to provide a safer methodology for collecting aqueous humor samples from the eye of a subject by providing stepwise draws upon a syringe plunger.

### BRIEF DESCRIPTION OF THE DRAWINGS

FIG. 1 is a drawing of the instrument in a position prior to movement of the syringe plunger.

FIG. 2 is a drawing corresponding to the drawing of FIG. 1 after movement of the syringe plunger.

FIG. 3 is a drawing of one embodiment of the instrument.

FIG. 4 is a drawing of the reverse side of the instrument relative to the view in FIG. 4.

FIG. 5 depicts aspects of an instrument according to embodiments of the present invention.

### DETAILED DESCRIPTION

Disclosed is an instrument used in collecting fluid samples from a subject. In some examples, the disclosed instrument comprises a linear actuator, a switch, a first syringe holder, a second syringe holder, and a housing.

In embodiments, the instrument comprises a linear actuator and a switch. The switch allows a user to engage the linear actuator such that one engagement of the switch by the user results in the linear actuator moving a defined distance. Further, the switch is configured such that the user can hold the instrument in a first hand and operate the switch with a second hand provided that the first hand is at the end of an extended arm of the user and the second hand is at the end of a flexed arm of the user. The first syringe holder is configured to accept all or a portion of a syringe plunger such that movement of the linear actuator by the defined distance moves the plunger the defined distance. The second syringe holder is coupled to a housing and is configured to accept a portion of a syringe barrel. The second holder is held immobile relative to the plunger such that a defined amount of fluid sample is collected in the syringe barrel when the syringe plunger moves the defined distance. The linear actuator is further configured to move the first holder away from the second holder upon engagement of the switch by the user.

The linear actuator can be any mechanism that results in linear motion of the first syringe holder (the syringe holder that engages the syringe plunger and that is coupled to the linear actuator) relative to the second syringe holder (the syringe holder that engages the syringe barrel and is coupled to the housing) in a stepwise manner. Linear actuators can include components such as a worm drive, a stepper motor, a DC motor, a lead-screw, a ball screw, a rack and pinion, or a ratchet/pawl mechanism or any of these alone or in combination with any other linear actuator component or any other mechanism that results in the linear movement of the linear actuator a defined distance.

The switch can be any mechanism that transmits a signal to the linear actuator to move the linear actuator the defined distance upon engagement of the switch by the user. In some embodiments, one engagement of the switch results in one movement of the linear actuator by the defined distance. The switch is also configured such that it can be operated by the second hand of a user holding all or a portion of the housing of the instrument in a first hand. In an example, the user can hold the housing of the instrument in a left hand and operate the switch with the right hand. In embodiments, the instrument is configured such that the user can operate other equipment, such as a slit lamp, microscope, camera, or other instrument used in visualization with the same hand as operates the switch. For example, the user can hold the housing of the instrument in the right hand and operate the switch and a slit lamp with the left hand. In some examples, the switch transmits the signal through a cable such as an electrical cable, a cable release, a control cable, a choke cable, or a throttle cable (the concept of transmitting a signal also encompasses mechanically transmitted signals). In other examples, the signal is transmitted wirelessly through infrared, Bluetooth®, visual light, or other wireless transmissions. The user engagement element of the switch itself can take any of a number of forms and a designer of ordinary skill in the art can select an appropriate design. Appropriate user engagement portions of switches can include but need not be limited to push buttons, toggle switches, roller switches, rocker switches, heat sensitive switches, touchscreens, plunger switches, or and any other switch that is attached to the instrument, separate from the instrument, or placed on other equipment (such as a slit lamp).

The first holder comprises a first portion configured to hold a plunger of a syringe. This first portion can permanently or (preferably) detachably hold the plunger in any position of the plunger such as at a proximal or (preferably) a distal end of the plunger. Any of a number of devices can be used for this purpose including a U-shaped or cylindrical device into which the plunger snaps into place, or any other such similar mechanism known in the art. Alternatively, the first portion of the holder can be adhered to or made to form all or part of the plunger. The first holder also comprises a second portion that is coupled to the linear actuator. This second portion can be (preferably) permanently or detachably coupled to a part of the linear actuator such that the first holder is configured to move relative to the second holder which holds the barrel of the syringe thereby pulling the plunger away from the needle and collecting sample within the syringe. Any of a number of mechanisms or devices can be used for this coupling. The second portion can be adhered to the linear actuator, formed as part of the linear actuator, or attached by a fastener (such as a screw, bolt, nut and bolt or other fastener,) or held in place via a U shaped or cylindrical holder on either the linear actuator or the first holder. In another alternative, the first holder can be configured to hold a plunger with an expanded circular tip. In this alternative, the first holder fits loosely or snaps around the main body of the plunger but fits under the expanded circular tip, thereby pulling the plunger via the expanded tip.

The second holder comprises a first portion configured to hold a barrel of a syringe. This first portion can permanently or (preferably) detachably hold the barrel in any position of the barrel. Any of a number of devices can be used for this purpose including a U-shaped or cylindrical device into which the barrel snaps into place or any such similar mechanism known in the art. Alternatively, the first portion of the holder can be adhered to or made to form all or part of the syringe barrel. The second holder also comprises a second portion that is coupled to the housing of the instrument. This second portion can be (preferably) permanently or detachably coupled to a part of the housing such that it is configured to not move relative to the housing. Any of a number of mechanisms or devices can be used for this coupling. The second portion can be adhered to the housing, formed as part of the housing, or attached by a fastener (such as a screw, bolt, nut and bolt or other fastener,) or held in place via a U shaped or cylindrical holder on either the linear actuator or the first holder.

The housing can be any fixed structure around the moving parts of the instrument (such as the linear actuator) that does not move during movement of the linear actuator and the first holder. The housing can fully or partially encase the moving parts of the instrument and can be made of any appropriate material including a plastic or a metal or combinations thereof. The housing can be of any shape including a shape that makes the instrument more easily held in the hand of the user such as a shape that includes a handle, ring, or other component known in the art that renders the instrument easier to hold.

In some examples, the instrument comprises an external power source such as a battery and/or can plug into a wall outlet. In such examples, the linear actuator can be powered by a motor such as a stepper motor. Such linear actuators can also comprise a rack and pinon, worm gear, or other mechanisms known in the art to provide linear motion.

In other examples, the linear actuator lacks an exterior power source and the movement of the linear actuator is provided by the energy exerted by the user in engaging the switch. In such an example, the switch is a mechanical switch such a mechanical cable release. Such a mechanical switch engages the linear actuator through extension of a telescoping or other extendable cable that is extended upon engagement of a switch (such as with a cable release). In the example of the cable release, the cable release can be of sufficient length that the user can hold all or a portion of the instrument in one hand and operate the switch with the other hand where the first hand is at the end of a substantially extended arm and the second hand is at the end of a substantially flexed arm. In this example, the cable can be at least 10 cm, at least 15 cm, at least 20 cm, at least 25 cm, at least 30 cm, at least 40 cm, at least 50 cm, at least 75 cm, at least 100 cm, or more than 100 cm in length.

In still other examples where the linear actuator lacks an exterior power source, the instrument can comprise an access to the linear actuator (comprising, for example, a ratchet/pawl mechanism) into which a mechanical switch (such as a cable release) can be permanently or temporarily inserted such that the cable release can be provided separate from the instrument (as in a kit) or as a replaceable component.

In some examples, the instrument is sufficiently light that it can be comfortably held in a substantially extended arm by the user for at least one minute, at least two minutes, at least three minutes, at least four minutes, at least five minutes, at least six minutes, at least seven minutes, at least eight minutes, at least nine minutes, or at least ten minutes. In still other examples, the instrument weighs 100 g or less, 90 g or less, 80 g or less, 70 g or less, 60 g or less, 50 g or less, 45 g or less, 35 g or less, 30 g or less, 25 g or less, 20 g or less, 15 g or less, 10 g or less, 5 g or less, 3 g or less, or 1 g or less.

In some examples, engagement of the switch by the user results in the first holder moving a defined distance, thereby moving the plunger of the syringe a defined distance relative to the barrel of the syringe. This distance is sufficient to draw a defined amount of fluid sample into the barrel of the syringe. How much fluid is drawn into the barrel depends on the nature of the fluid sample intended to be drawn and one of skill in the art in light of this disclosure could modify the disclosed instrument to draw any of a number of volumes of fluid. In one example, the fluid sample to be drawn comprises aqueous humor from the anterior chamber of the eye—the fluid occupying the area between the cornea and lens. The total volume of the space is about 250 μl, so removal of more than 150 μl could result in complications. As a result, removal of a defined volume per engagement of the switch for this particular use could range from 1-100 μ1, 5-50 μl, 10-30 μl, 10-20 μl, or any range therein. Absolute reproducibility of the defined volume per engagement of the switch is not necessary from one engagement to the next and one of skill in the art can understand that a range of volumes per engagement in the instrument can be tolerated so long as they do not vary sufficiently to result in the unexpected removal of more than a maximal safe volume.

In still further examples, the instrument comprises a reset mechanism. The reset mechanism can move the first holder to a starting position. The starting position for the first holder can be anywhere that the first holder can hold the plunger of the syringe while the second holder can hold the barrel of the syringe and where engagement of the switch by the first user can still result in the drawing of liquid into the syringe. Preferably, the starting position places the distal end of the plunger (the portion of the plunger furthest from the first holder) at or near the distal end of the barrel (the portion of the barrel nearest the needle). In the starting position, the plunger can be in contact with, within 1 mm, 2 mm, 3 mm, 5 mm, 7 mm, 10 mm of or more than 10 mm away from the distal end of the barrel. One of skill in the art in light of this disclosure can design an appropriate reset mechanism. For example, a reset mechanism includes a mechanism that allows disengagement of the stepper motor from the linear actuator and/or disengagement of the ratchet/pawl mechanism allowing movement of the first holder back towards the second holder. Preferably, the sample and/or the syringe is removed from the instrument prior to engagement of the reset mechanism.

### EXAMPLES

The following examples are illustrative of disclosed methods. In light of this disclosure, those of skill in the art will recognize that variations of these examples and other examples of the disclosed method would be possible without undue experimentation.

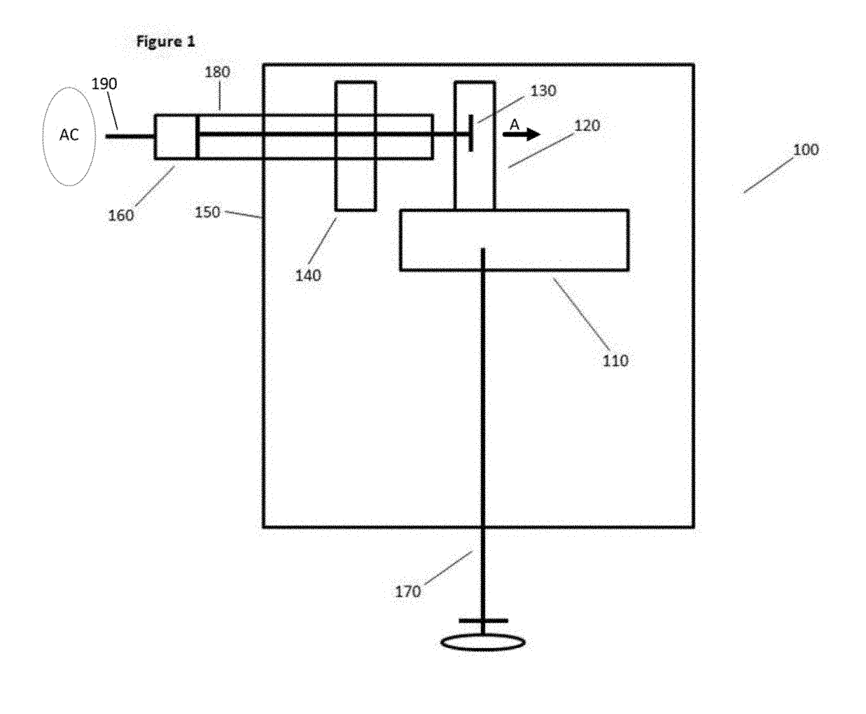

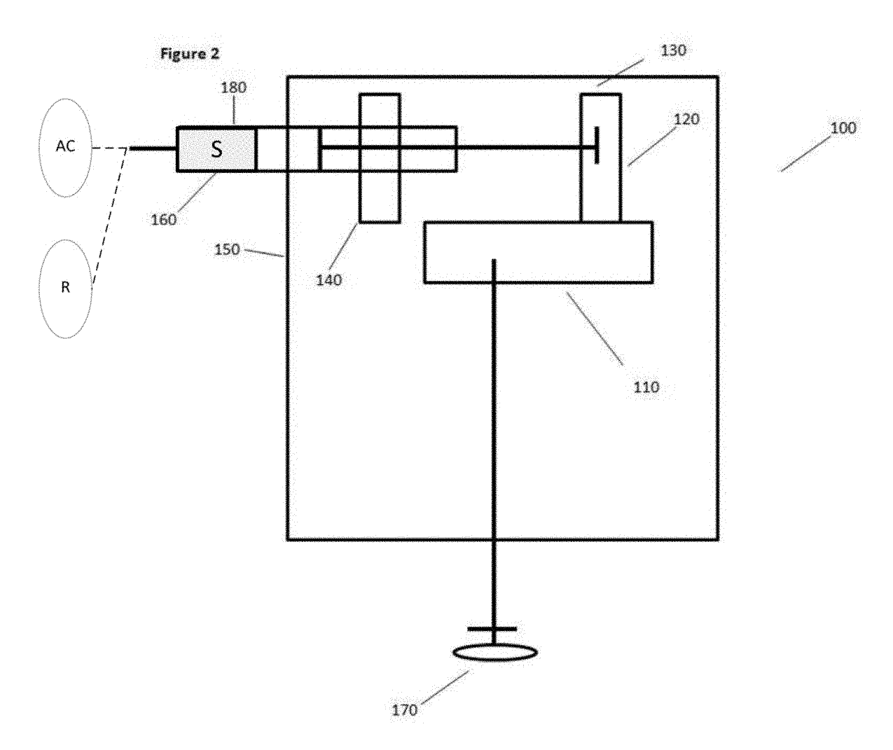

Referring now to FIG. 1, the instrument, 100 comprises a linear actuator 110 and a first holder 120 coupled to the linear actuator 110. The first holder holds the plunger 130 of the syringe 180. The instrument further comprises a second holder 140 that is coupled to a housing 150 where the second holder 140 is configured to hold the barrel 160 of the syringe 180. Upon engagement of the switch 170, the linear actuator 110 causes the first holder 120 to move away from the second holder 140 in the direction indicated by arrow A thereby pulling the plunger 130 to draw the sample from an anterior chamber AC of a patient eye into the barrel 160 of the syringe 180 via a syringe needle 190. FIG. 2 shows the instrument 100 after engagement of the switch 170. Note that the first holder 120 has moved relative to the second holder 140, and fluid sample S has been drawn into barrel 160 of syringe 180. Although certain embodiments of the present invention describe the use of instrument 100 for obtaining a sample of aqueous humor from an anterior chamber AC of an eye of a patient, it is understood that in other embodiments the instrument 100 can be used to obtain samples (e.g. fluid) from other locations within the body of a patient, for example any reservoir R of a patient body that contains or holds a fluid.

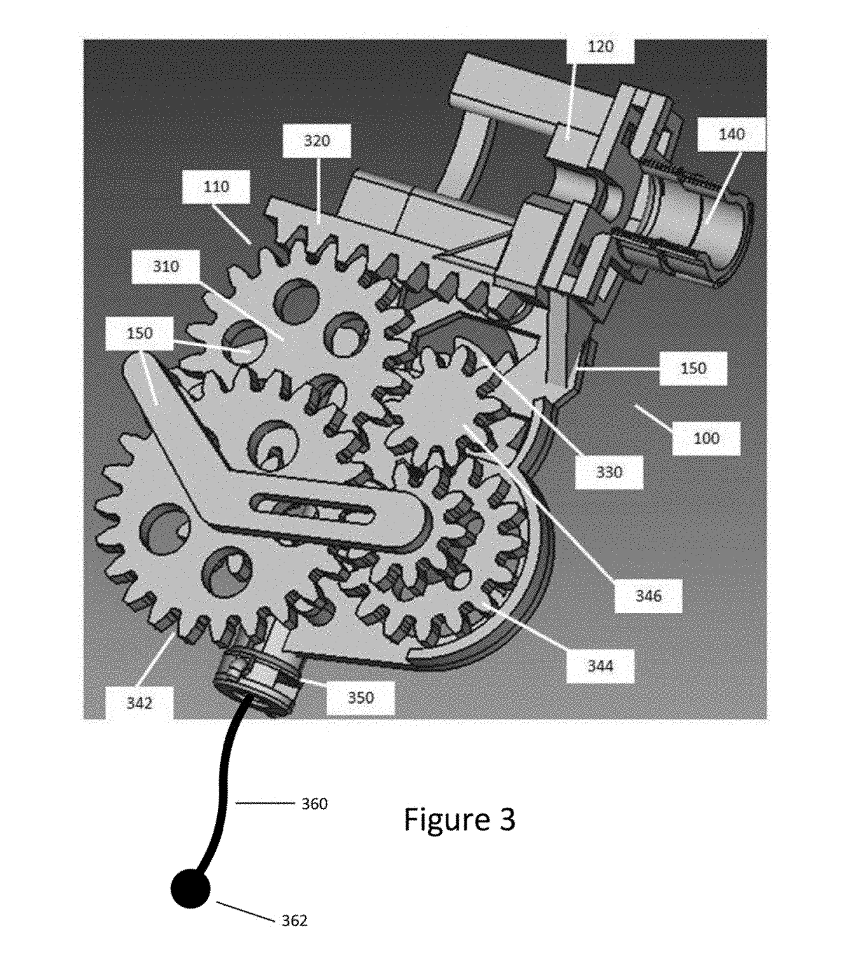

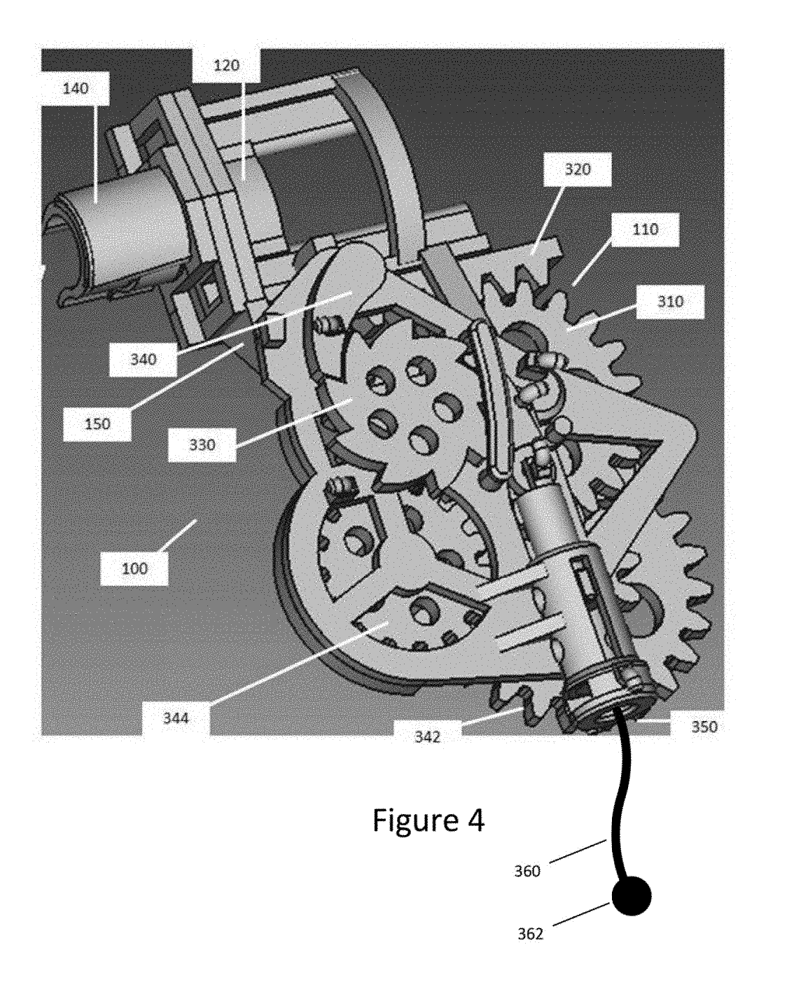

Referring now to FIG. 3, one example of the instrument 100 comprises a linear actuator 110. In this example, the linear actuator is in the form of a rack and pinion mechanism comprised of a first circular gear (the pinion) 310 and a linear gear (the rack) 320. A ratchet 330 and pawl mechanism 340 (the pawl mechanism 340 is not visible in FIG. 3 but is shown in FIG. 4) is incorporated into the instrument 100 to ensure unidirectional motion of the linear actuator during operation. The linear gear 320 is coupled to the first holder 120 while the second holder 140 is coupled to the housing 150. An access 350 for a cable release 360 can engage the cable release with a telescoping interior cable (not shown) and engage the ratchet pawl mechanism 340 thereby moving a second circular gear 342, a third circular gear 344, a fourth circular gear 346, and the first circular gear 310 (the pinion), to translate the linear gear 320 (the rack) and first holder 120. In some instances, the cable release 360 can engage directly with the ratchet pawl mechanism 340. According to some embodiments, a cable release 360 can include an actuator or plunger 362, and during use each actuation (e.g. push or pull) of the actuator or plunger 362 can operate to activate the ratchet pawl mechanism 340. In some cases, the cable release can include a semi-rigid plastic or braided metal outer tube, through which an inner metal wire passes. The inner metal wire can be coupled with a cylindrical piece of metal that can extend from or retract into a distal end of the outer tube, so as to engage and actuate the ratchet pawl mechanism 340. FIG. 4 is a view from the opposite side of the instrument as FIG. 3.

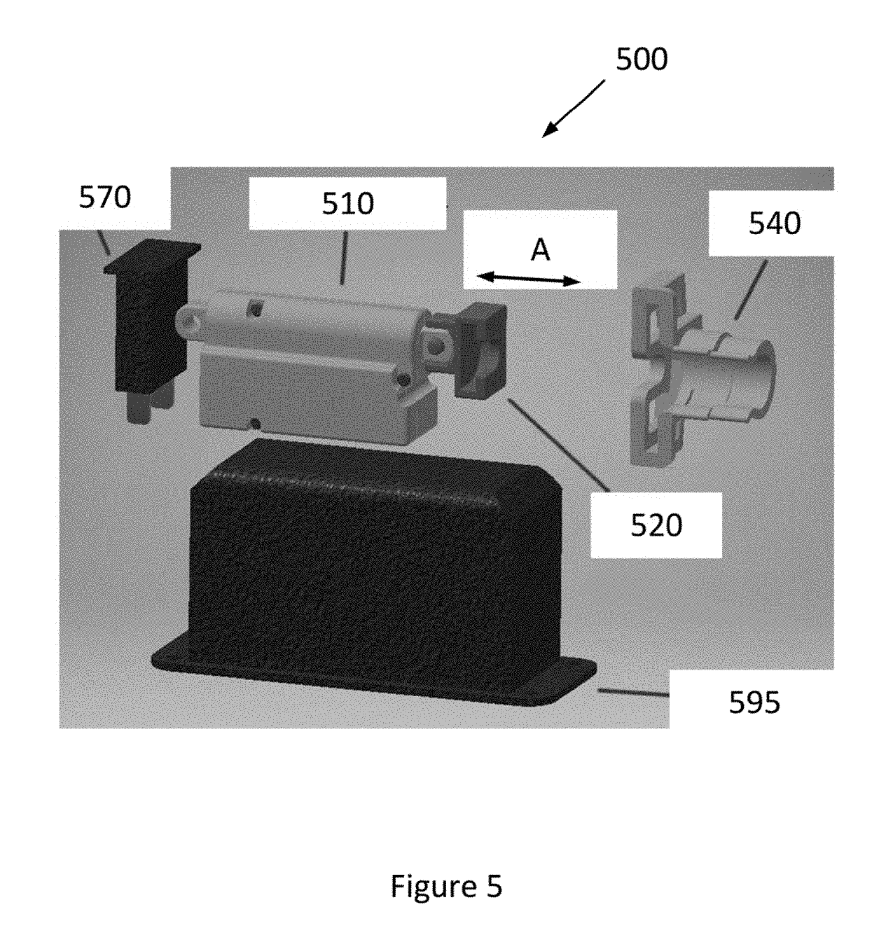

FIG. 5 depicts aspects of an instrument 500 according to embodiments of the present invention. The instrument includes a housing (not shown, which may be in the form of a box, for example), a syringe (not shown), a linear actuator 510, a first holder 520 (e.g. configured to hold a plunger of the syringe) coupled with the linear actuator 510, a second holder 540 (e.g. configured to hold a barrel of the syringe) coupled with the housing, and a switch 570, which in some instances may be an on/off switch. In use, the instrument 500 can be operated by a physician or other surgical personnel to collect a fluid sample from a patient. For example, the instrument 500 can be operated to collect a sample of aqueous humor from an anterior chamber of an eye of a patient. The switch 570 can be configured to allow the user to engage the linear actuator 510 such that one engagement of the switch 570 by the user causes the linear actuator 510 to move a defined distance thereby moving the first holder 520 (e.g as indicated by arrow A) a defined distance, which in turn moves the syringe plunger a defined distance. The second holder 540 is held immobile or otherwise remains fixed while the plunger moves. In this way, a defined amount of the fluid sample can be collected in the syringe barrel when the syringe plunger moves the defined distance. Instrument 500 represents an electronic version of a fluid collection device, and can be used with a variety syringe types, including 1 mL syringes, for example, which can be snapped into place or otherwise engaged with the first holder 510 and the second holder 540. In FIG. 5, the linear actuator 510 is depicted in a retracted state. In use, at the onset of operation, the linear actuator 510 can instead be in an extended state. In some cases, with each press of the control button or switch 570, the linear actuator 510 can retract by an incremental amount, so as to draw incremental amounts of sample into the syringe. As shown here, the instrument 500 can also include a power source 595 to provide power to the linear actuator 510. In some cases, the power source 595 may include a 9 volt battery, in a box or housing. In some cases, a power source 595 can include a source of electrical power, such as a battery or AC current.

According to some embodiments, the instrument 500 may include a control box (not shown). The control box can include a small box (that can be held with the other hand that is not holding the instrument housing) and can be connected to the main hand piece or instrument housing by a flexible cord (e.g. about 18 inches long). The control box can contain a small circuit board, an LED indicator to show that the device is on, and a single button to actuate the device. According to some embodiments of an electronic version of the instrument, the control box may also include a small speaker or buzzer. The speaker or buzzer can be configured to emit a simple tone for the duration of each incremental actuator movement to alert the user when each discrete movement is complete. In some cases, the linear actuator may make enough noise that it will be audibly apparent to the user when the device is moving.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An instrument used in collecting fluid samples from a subject, the instrument comprising:
a linear actuator;
a switch configured to allow a user to engage the linear actuator such that one engagement of the switch by the user causes the linear actuator to move a defined distance;
a first holder coupled to the linear actuator where the first holder is configured to accept a portion of a syringe plunger such that the movement of the linear actuator the defined distance moves the syringe plunger the defined distance; and
a second holder coupled to the housing, where the second holder is configured to accept a portion of a syringe barrel and where the second holder is held immobile relative to the plunger such that a defined amount of the fluid sample is collected in the syringe barrel when the syringe plunger moves the defined distance.

2. The instrument of claim 1 comprising an electrical power source.

3. The instrument of claim 1 where the linear actuator comprises a worm gear, lead screw, or ball screw and where the switch controls a stepper motor, servo motor, or DC motor.

4. The instrument of claim 1 where the switch engages the linear actuator through a mechanical cable release, an electrical cable, or an infrared beam.

5. The instrument of claim 1 where the instrument lacks an electrical power source.

6. The instrument of claim 1 where the linear actuator comprises a rack and pinion and where the switch mechanically engages a ratchet/pawl mechanism.

7. The instrument of claim 1 where the instrument weighs 50 g or less.

8. The instrument of claim 1 where the defined amount of fluid sample is 5-50 μl.

9. The instrument of claim 8 where the defined amount of fluid sample is 10-30 μl.

10. An instrument used in collecting fluid samples from a subject, the instrument comprising:
a linear actuator comprising a linear gear and a first circular gear;
a ratchet/pawl mechanism engaged with the first circular gear;
an access to the ratchet/pawl mechanism configured to accept a cable release such that the cable release can engage with the ratchet/pawl mechanism;
a first holder coupled to the linear gear where the first holder is configured to accept a portion of a syringe plunger; and
a second holder coupled to a housing, where the second holder is configured to accept a portion of a syringe barrel and where the second holder is held immobile relative to the first holder;
where the linear actuator is configured to increase the distance between the first holder and the second holder upon engagement of the switch by the user.

11. The instrument of claim 10 where the cable release is coupled to the access.

12. (canceled)

13. The instrument of claim 10 where the cable release is at least 10 cm in length.

14. The instrument of claim 10 further comprising a second circular gear engaged with the ratchet/pawl mechanism and the first circular gear.

15. The instrument of claim 1 further comprising a reset mechanism configured to return the second holder to a starting position.

16. A kit used in the collection of a fluid sample from a subject, the kit comprising:
the instrument of claim 1; and
a syringe comprising a plunger configured to engage the first holder, a barrel configured to engage the second holder, and a needle.

17. The kit of claim 16 further comprising a cable release, where the cable release is configured to engage the access of the instrument of claim 10.

18. The kit of claim 16 where the needle is 25 gauge or smaller.

19. A method of collecting a sample comprising aqueous humor from an anterior chamber of an eye of a subject, the method comprising:
inserting a needle into the anterior chamber, where the needle is attached to a syringe, the syringe further comprising a barrel and a plunger, where the plunger of the syringe is held by the first holder of the instrument of claim 1 and where the barrel of the syringe is held by the second holder of the instrument of claim 1;
engaging the switch of the instrument of claim 1, thereby collecting the sample comprising aqueous humor.

20. The method of claim 19 further comprising inserting the syringe into the instrument of claim 1.

21. The method of claim 19 where the instrument is held by a first hand of a user and where the switch is operated by a second hand of the user.

22. The method of claim 21 where the second hand of the user is used to operate a slit lamp in addition to operating the switch.

23. The method of claim 19 further comprising engaging the switch a sufficient number of times to collect at least 100 μl of the sample.

24. The method of claim 19 further comprising removing the syringe from the instrument and resetting the first holder to a starting position.
